(19) United States
(12) Patent Application Publication
(10) Pub. No.: US 2002/0151593 A1
(43) Pub. Date: Oct. 17, 2002

(54) Water-soluble creatine monohydrate formulations and process for their preparation

(76) Inventors: James Stitley JR., West Jordan, UT (US); Anthony Faranetta, Sandy, UT (US)

Correspondence Address: NIXON & VANDERHYE P.C., 1100 North Glebe Road, 8th Floor, Arlington, VA, 22201, US

(21) Appl. No.: 09/833,800

(22) Filed: Apr. 13, 2001

(51) Int. Cl. A61K 31/198

(52) U.S. Cl. 514/565

(57) ABSTRACT

A process for preparing a water-soluble creatine monohydrate, comprising blending creatine monohydrate having an average particle size of 40 microns lor less and a water soluble agglomeration prevention agent, such as dextrose.

The present invention relates to water-soluble creatine monohydrate formulations. More particularly, the present invention provides a creatine monohydrate powder that is substantially completely soluble in cool water and dissolves in less than 5 minutes. A process for preparing the formulations of the invention is also provided.

### BACKGROUND OF THE INVENTION

Creatine (N-(aminoiminomethyl)-N-methylglycine) is a sarcosine derivative which is produced naturally in humans and other animals. It is produced in the liver, kidney and pancreas, and is supplied to the body by the food intake. Creatine is converted to creatine phosphate in the muscles, and the creatine phosphate is stored in the muscle as an available source of phosphate for the resynthesis of adenosine triphosphate (ATP) from adenosine diphosphate. Creatine is primarily combined with phosphoric acid in the form of phosphoryl creatine. Muscle fatigue and the accumulation of lactic acid occur when the supply of phosphoryl creatine is exhausted and the adenosine diphosphate cannot be converted to adenosine triphosphate.

Studies suggest that there is a relationship between the creatine (phosphoryl creatine) concentration in the muscles having the function of keeping a high intracellular ATP/ADP ratio and maximum sustainable physical effort. Creatine is normally present in the bloodstream at a concentration of about 50 mol per liter of blood. Increasing the amount of creatine within muscle is believed to favorably affect muscular performance and the amount of work which can be done by the muscle.

Increasing the amount of creatine in diets may therefore be useful to elevate the plasma creatine concentrations to levels providing significant benefit of creatine in the muscle. However, the short creatine half-life in plasma (1-1.5 hours) makes it necessary to reach such levels rapidly. Increasing the level of creatine in the bloodstream is generally achieved by administration of high doses (5-10 g for mean body weights of 70 kg) of creatine, which are well tolerated because of the lack of toxicity of creatine.

Creatine is only sparingly soluble in water (1 g in 75 ml at ambient conditions) and so the amount of creatine that can be supplied in solution is limited. This low solubility in water is a practical limitation to the possibility of making immediately available in specific diets the necessary amounts of creatine.

Creatine is generally not effectively administered orally in powder form, since creatine rapidly converts to creatinine in the acidic conditions in the stomach, and is not in a soluble bioavailable form causing a disturbance in the positive osmotic pressure necessary for absorption. Creatinine is the inactive form of creatine and is quickly depleted from the body. Moreover, creatinine is not able to convert to creatine phosphate and does not participate in the regeneration of adenosine triphosphate.

Creatine as received from a supplier is typically insoluble in cool tap or spring water at about 13° C. Drinks containing undissolved creatine particles are found to have an objectionable texture (sandy), and sediment formation occurs.

Attention has focused recently on the benefits of creatine monohydrate. The monohydrate makes the creatine more bioavailable and is an important vehicle for administration of creatine. The Merck Index indicates that creatine monohydrate is soluble in water in a ratio of 1 gram of monohydrate to 75 ml water. However, the water referred to is distilled water, and is at room temperature (20° C.). Distilled water at 20° C is not readily available in homes, work-places, gymnasiums and elsewhere. Drinking water (tap water and drinking fountain water) is typically at a lower temperature, in the region of 12-15° C. Currently, there are no commercially available creatine monohydrate products which are completely and rapidly soluble in cool (approx. 13° C.) water.

A need exists for a storage stable completely water-soluble creatine monohydrate formulation which dissolves rapidly in cool water. The present invention seeks to fill that need.

### SUMMARY OF THE INVENTION

It has been discovered, surprisingly, according to the present invention, that it is possible to provide creatine monohydrate in a form which is completely soluble in cool water within 5 minutes or less. This enables the preparation of creatine monohydrate formulations which can be completely dissolved in cool water and drunk as a clear solution in one serving in the absence of undissolved suspended or precipitated powder.

According to one aspect of the invention, there is provided a process for preparing a water-soluble creatine monohydrate, comprising blending creatine monohydrate having an average particle size of 40 microns or less and a water soluble agglomeration prevention agent.

In a further aspect, there is provided a water-soluble creatine monohydrate produced by the process of the invention.

In another aspect, there is provided a storage stable water-soluble powder comprising creatine monohydrate with an average particle size of 40 microns or less and an agglomeration prevention agent.

In yet another aspect, there is provided an aqueous formulation comprising creatine monohydrate with an average particle size of 40 microns or less and an agglomeration prevention agent.

In a further aspect, there is provided an aqueous composition comprising 5 grams of creatine monohydrate and an agglomeration prevention agent dissolved in 18 ounces of water (one serving).

### DETAILED DESCRIPTION OF THE INVENTION

The process of the invention provides for the preparation of a water-soluble creatine monohydrate which dissolves in cool (12-15° C.) water in less than 5 minutes, more usually in less than 1 minute and typically in less 10 seconds, to provide a palatable drinkable creatine monohydrate solution. The process comprises blending creatine monohydrate having an average particle size in the range of 40 microns or less, for example 10 microns or less, more usually 1-10 microns, and an excess of a particulate water soluble agglomeration prevention agent together. An agglomeration prevention agent is employed because it has been found, during development of the present invention, that creatine monohydrate particles tend to recombine or agglomerate during and after grinding. The presence of a particulate water soluble agglomeration prevention agent substantially reduces the tendency of creatine monohydrate to agglomerate during grinding.

As used herein, the term “agglomeration prevention agent” means a substance which reduces or eliminates the tendency of creatine monohydrate to agglomerate as the particle size of the creatine monohydrate is reduced, for example during grinding. The agglomeration prevention agent is typically in particulate form and is blended with the creatine monohydrate in a blender.

Without being bound to any theory, it is believed that the tendency of particulate creatine monohydrate to agglomerate is reduced in the presence of the water soluble agglomeration prevention agent as a result of the creatine monohydrate being plated or coated at least in part on the surface of the particles of the agglomeration prevention agent. The agglomeration prevention agent is provided in an excess of the creatine monohydrate, typically at least four times the weight excess of the creatine monohydrate, to ensure that all creatine monohydrate is plated onto the particulate agglomeration prevention agent. Examples of suitable water soluble agglomeration prevention agents are carbohydrates, including sugars, for example dextrose and sucrose. Alternatively, it is possible to use as the agglomeration prevention agent a soluble fiber material.

The term “excess”, as used herein in relation to the amount of agglomeration prevention agent, means a weight excess over the weight of creatine monohydrate present, to allow all of the creatine monohydrate to be plated on the surface of the agglomeration agent to minimize the tendency of the creatine monohydrate to agglomerate or combine during grinding. Typically, the agglomeration prevention agent is present in a weight excess of 1:2-1:6 creatine monohydrate: agglomeration prevention agent, more usually 1:4.

The term “plating” as used herein means the layering or coating of creatine monohydrate on the surface of the agglomeration prevention agent. Plating occurs during grinding of the creatine monohydrate with the agglomeration prevention agent when creatine monohydrate is brought into intimate contact and worked with the agglomeration prevention agent.

The creatine monohydrate is ground using a mill specially designed for ultra-fine grinding, (<10 microns). An example of such a mill is a Quadro Mill equipped with a screen having 0.018 inch diameter holes (450 microns). The mill employs air to propel the particles to sonic velocities causing the particles to grind themselves via attrition. This unique principle achieves ultra-fine particles without imparting heat (creatine monohydrate is heat liable). Also, the design of the mill allows for classifying the particles. Only particles meeting a predetermined particle size range will exit the mill.

Grinding is carried out in the substantial absence of water to reduce the risk of premature dissolution and loss of product. It is generally not necessary to introduce grinding aids.

The agglomeration prevention agent, such as dextrose, is typically ground in a conventional grinder (for example a Quadro Mill (0.018 screen)), followed by the creatine monohydrate to produce ground dextrose having an average particle size of 250 microns to 75 microns, more usually 200-150 microns, for example 177 microns to 150 microns. This may be achieved by grinding through a Quadro Mill equipped with a screen having 0.018 inch diameter holes (450 microns) and operating at 1800 rpm's. The rpm's can be varied from 500 to 3700, depending on the agglomeration prevention material being used. Moreover, the speed is selected to minimize introduction of heat to either of the materials. This process produces an agglomeration prevention agent within an acceptable particle size range.

The ground creatine monohydrate and the ground agglomeration prevention agent are blended to produce a blend in which creatine monohydrate is uniformly distributed with the ground agglomeration prevention agent. Blending may be achieved using any conventional blender, such as for example a V-Blender. Blending typically takes of the order about 15 minutes, more usually 10-20 minutes to achieve a homogeneous mixture.

The homogeneous mixture of creatine monohydrate and the agglomeration prevention agent is then typically subjected to a further grinding step to produce a finely ground blend of creatine monohydrate plated on the agglomeration prevention agent. The blend particles have a size range of 1 to 200 microns.

The invention further provides a storage stable water-soluble powder comprising creatine monohydrate and an excess of an agglomeration prevention agent. The average particle size of the powder is 1 to 200 microns. The creatine monohydrate and the agglomeration prevention agent are typically present in a weight ratio range of creatine monohydrate:agglomeration prevention agent of 1:10 to 1:2, for example 1:4, with the creatine monohydrate plated on the surface of the agglomeration prevention agent. The creatine monohydrate typically has a particle size in the range of about 2.0 microns (Std. Deviation=4.8 microns).

The grinding and blending steps of the process of the invention are generally carried out at ambient (room) temperature. Creatine monohydrate, when heated, tends to convert to creatinine which is undesirable. The temperature of the process is generally controlled to ensure that the temperature does not exceed 25-28° C.

The powder blend may additionally comprise conventional coloring agents and flavoring agents. These agents may be incorporated during blending and/or the final grinding steps.

The powder blend of creatine monohydrate and agglomeration prevention agent dissolves in water at a temperature of 12-15° C. within 1 minute, more usually within 10 seconds, often less than 5 seconds with shaking. The powder blend may be quickly and conveniently formulated in cool tap water or cool drinking fountain water to form a palatable drink, with substantially no sediment or cloudiness.

An aqueous formulation is also provided comprising creatine monohydrate and an agglomeration prevention agent. The aqueous formulations of the invention exhibit good shelf-storage properties and do not produce sediment on standing.

In a particular aspect, there is provided, as a single serving, an aqueous composition comprising about 5 grams of creatine monohydrate and an agglomeration prevention agent dissolved in 18 ounces of water. This dosage in water is close to the point of maximum solubility of creatine monohydrate.

Most commercially available products, under typical time, temperature and concentration conditions, have an unacceptable fallout or precipitation of larger particles at the bottom of the drinks, commonly referred to as “wet sand”, which is unacceptable to the consumer. The human eye typically can detect discrete particles above 40 microns, and not below 40 microns. In the present invention, the creatine (monohydrate) particles are ground to a size at or smaller than 40 microns such that, even if not in full solution, the liquid drink still would not have sentiment on the bottom but might, in a worst case, have a slightly cloudy appearance from suspended but perhaps undissolved particles at this particle size and the 5 gram concentration.

It is well known in that a finer grind particle size will increase solubility, in general. Since 5 grams is close to the threshold, the less than 40 micron median particle size may be more soluble. Experimentation has proved this out coupled with the addition of an agglomeration prevention agents, such as a carbohydrate water soluble agglomeration prevention agent, to increase solubility.

Subsequent particle grinding reduces the particle size well below the eye detection limit of 40 microns. When 5 grams of finely reduced creatine with a carbohydrate water soluble agglomeration prevention agent is added to water, the active ingredient goes substantially immediately into solution and does not form a cloudy suspension.

### EXAMPLES

The invention will now be described with reference to the following working examples, in which ratios are by weight.

### Example 1

Creatine monohydrate was ground through a Quadro Mill equipped with a screen having 0.018 inch diameter holes (450 microns). This reduced the particle size but not sufficient to achieve solubility. This ground product was then sifted through a 325 mesh (44 microns) and a 400 mesh (37 microns) to isolate smaller particle sizes. In both cases, it appeared that some of the creatine monohydrate particles were agglomerating and becoming insoluble.

To overcome this agglomeration, dextrose was ground through the Quadro Mill (0.018 screen), followed by the creatine monohydrate. This produced ground dextrose having an average particle size range of 250 microns to 75 microns, and ground creatine monohydrate having an average particle size range of 40 microns or less. The ground creatine monohydrate and ground dextrose were combined in a weight ratio of 1:4, and the resulting mixture was re-ground through the Quadro Mill (0.018 screen) to produce a creatine monohydrate/dextrose blend having an average particle size range of 1-200 microns. The objective was to maximize the number of dextrose particles with the first grinding and then, using the Quadro, to plate the creatine monohydrate onto the dextrose thereby keeping the creatine monohydrate particles separated and preventing re-agglomeration of the creatine monohydrate. This procedure improved the rate at which the creatine monohydrate became soluble, but not in all cases. It was found that the blend of creatine monohydrate and dextrose was not homogenous and therefore the creatine monohydrate concentration in the final product varied. Solubility is dependent on concentration.

The creatine monohydrate was then ground to a smaller particle size by grinding to an average particle size of 6.5 microns (S.D.=4.8 microns). This finely ground creatine monohydrate was combined with dextrose (1:4 weight ratio) which had been ground through the Quadro Mill (0.018 screen) and had an average particle size range of 40 microns or less and blended in a V-Blender. The resulting creatine monohydrate/dextrose blend was re-ground through the Quadro Mill with the 0.018 screen to produce a finely ground creatine monohydrate/dextrose blend having an average particle size range of 1 to 200 microns. This finely ground blended material was completely soluble when combined with 18 oz of 55° F. water and shaken in a bottle for 15 seconds.

### Example 2

Individual samples of the ground creatine monohydrate having a particle size of about 6.5 microns and ground dextrose before V-blending were combined in the weight ratio 1:4. The resulting blends were substantially insoluble.

### Example 3

The dextrose particle size was further reduced to approximately the same size as the creatine (6 microns). The creatine and dextrose were then blended in a weight ratio of 1:4 and re-ground to the smallest particle size attainable. The resulting blend was instantly soluble (within 10 seconds with some shaking or gentle shaking).

### Example 4

The dextrose particle size is reduced to approximately the same size as the creatine (6 microns), and the creatine and dextrose are blended in a weight ratio of 1:10 and re-ground to the smallest particle size attainable. The resulting blend is instantly soluble (within 10 seconds with some shaking or gentle shaking).

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

What is claimed is:

1. A process for preparing a water-soluble creatine monohydrate, comprising blending creatine monohydrate having an average particle size in the range of 40 microns or less and a water soluble agglomeration prevention agent.

2. A process according to claim 1, wherein said creatine monohydrate is plated onto said agglomeration prevention agent.

3. A process according to claim 1, wherein said agglomeration prevention agent is present in excess.

4. A process according to claim 3, wherein said creatine monohydrate and said agglomeration prevention agent are blended in a weight ratio 1:4 creatine monohydrate:agglomeration prevention agent.

5. A process according to claim 1, wherein said creatine monohydrate has an average particle size of about 4-10 microns.

6. A process according to claim 1, wherein said creatine monohydrate has an average particle size of about 6.5 microns (S.D.=4.8 microns).

7. A process according to claim 1, wherein said agglomeration prevention agent has an average particle size in the range of 250 microns to 75 microns.

8. A process according to claim 1, wherein said agglomeration prevention agent is a carbohydrate, selected from the group consisting of dextrose and sucrose

9. A process according to claim 1, wherein said agglomeration prevention agent is a soluble fiber.

10. A process for preparing a water-soluble creatine monohydrate, comprising the steps of:
grinding creatine monohydrate to produce ground creatine monohydrate having an average particle size of less than 40 microns;
grinding a water soluble agglomeration prevention agent to produce ground agglomeration prevention agent having an average particle size of 250 microns to 75 microns; and
blending said ground creatine monohydrate and said ground agglomeration agent to produce a blend in which the agglomeration prevention agent is in excess of the amount of creatine monohydrate and in which said creatine monohydrate is plated on said agglomeration prevention agent.

11. A process according to claim 10, wherein said blend of creatine monohydrate and agglomeration prevention agent is ground to produce a finely ground blend of creatine monohydrate and agglomeration prevention agent.

12. A water-soluble creatine monohydrate prepared by the process of claim 1.

13. A storage stable water-soluble powder comprising creatine monohydrate with an average particle size of 40 microns or less and an agglomeration prevention agent.

14. A powder according to claim 13, wherein said creatine monohydrate and said agglomeration prevention agent are present in a weight ratio of 1:10 creatine monohydrate:agglomeration prevention agent.

15. A powder according to claim 13, wherein said creatine monohydrate and said agglomeration prevention agent are present in a weight ratio of 1:4 creatine monohydrate:agglomeration prevention agent.

16. A powder according to claim 13, wherein said creatine monohydrate has a particle size in the range of about 6.5 microns.

17. A powder according to claim 13, wherein said creatine monohydrate is plated on said agglomeration prevention agent.

18. A powder according to claim 13, wherein said agglomeration prevention agent is a carbohydrate selected from the group consisting of dextrose and sucrose.

19. A powder according to claim 13, which dissolves in water at a temperature of 12-15° C. within 1 minute.

20. A powder according claim 13, which dissolves in water at a temperature of 12-15° C. within 10 seconds.

21. A powder according to claim 13, further comprising a coloring agent and a flavoring agent.

22. An aqueous formulation comprising creatine monohydrate and an agglomeration prevention agent.

23. An aqueous composition comprising about 5 grams of creatine monohydrate and an agglomeration prevention agent dissolved in 18 ounces of water.
